(19) United States
(12) Patent Application Publication
(10) Pub. No.: US 2024/0374616 A1
(43) Pub. Date: Nov. 14, 2024

(54) USE OF TERPENOIDS IN THE TREATMENT OF MASTOCYTOMA

(71) Applicant: ARJIL BIOTECH HOLDING COMPANY LIMITED, Hsinchu City (TW)

(72) Inventors: Yeh B Wu, Hsinchu City (TW); Jir-Mehng Lo, Hsinchu City (TW); Hui Ju Liang, Taipei City (TW); Pei-Hsin Lin, Hsinchu County (TW)

(73) Assignee: ARJIL BIOTECH HOLDING COMPANY LIMITED, Hsinchu City (TW)

(21) Appl. No.: 18/691,255

(22) PCT Filed: Sep. 13, 2022
(86) PCT No.: PCT/US2022/076346

§ 371 Date: Mar. 12, 2024

Related U.S. Application Data
(60) Provisional application No. 63/243,392, filed on Sep. 13, 2021.

(51) Int. Cl. A61K 31/575; A61K 31/365; A61P 35/00

(52) U.S. Cl. CPC A61K 31/575; A61K 31/365; A61P 35/00

(57) ABSTRACT

The present invention relates to a method of treating mast cell tumor, comprising administering an effective amount of composition to a subject in need thereof: wherein the composition comprises terpenoids extracted from Antrodia camphorate or Anisomeles indica.

### FIELD OF THE INVENTION

The invention relates to herbal terpenoids from Antrodia camphorata and Anisomeles indica extract, particularly to a medicinal and edible formula to treat mast cell tumor effectively.

### BACKGROUND OF THE INVENTION

A mastocytoma or mast cell tumor is a type of round-cell tumor consisting of mast cells that occurs in many non-human animal species. If complete removal of the mast cell tumor through surgery is not possible due to the size or location, additional treatment, such as radiation therapy or chemotherapy, may be necessary. Vinblastine and lomustine are common chemotherapy agents used to treat dog mast cell tumors. Toceranib and masitinib, examples of receptor tyrosine kinase inhibitors, are used in the treatment of canine mast cell tumors. Both were recently approved by the U.S. Food and Drug Administration as dog-specific anticancer drugs. However, chemotherapeutic techniques have a range of adverse side effects that depend on the type of medications used. The use of medicinal plant-derived products to manage or arrest the carcinogenic process provides a safer alternative to the use of conventional chemodrug for treatment of the disease.

Medicinal fungus Antrodia camphorata (AC) is a well-known Chinese folk medicine, known to possess numerous biological activities. To date, a total of 225 compounds have been isolated, identified, and structurally elucidated, including macromolecules (nucleic acids, proteins, and polysaccharides), small molecules (benzenoids, lignans, benzoquinones, and maleic/succinic acid derivatives), terpenoids (lanostane triterpenes, ergostane triterpenes, diterpenes, monoterpenes, and steroids), nucleotides (nucleobase and nucleoside), fatty acids, and fatty acid esters.

Anisomeles indica commonly known as ‘Indian Catmint’ is a source of medicinally active compounds and have various pharmacological effects. The plant is used traditionally as an analgesic, anti-inflammatory and in skin problems. Further studies reveal the presence of various phytochemical constituents mainly triterpenes, β-sitosterol, stigmasterol, flavones, apigenin and ovatodiolides etc.

Plant terpenoids play a role in traditional herbal remedies and are also abundant in Antrodia camphorate and Anisomeles indica. However, the efficacy of these terpenoids and combination thereof in the treatment of mast cell tumor has not been evaluated.

### BRIEF DESCRIPTION OF THE DRAWINGS

FIG. 1 depict the effects of terpenoids on the cell viability on RBL-2H3 cells and the inhibition effect on β-hexosaminidase release.

FIG. 2 depicts the effects of terpenoids on the cell viability in RBL-2H3 cells.

FIG. 3 depicts the effects of terpenoids on the cell viability and the β-hexosaminidase release in RBL-2H3 cells.

FIG. 4 depicts the effects of terpenoids on the cell viability and the β-hexosaminidase release in RBL-2H3 cells.

FIG. 5 depicts the effects of combining terpenoids on P815 cell viability.

FIG. 6 depict the animals' body weight and tumor volume.

FIG. 7 depict the animals' tumor volume at 30th day.

FIG. 8 depict the animals' tumor weight at 30th day.

FIG. 9 depict the animals' tumor volume at 30th day.

FIG. 10 depict the photographs of tumor appearance at 23rd day.

FIG. 11 depicts H&E histological stain of tumor tissues.

FIG. 12 depicts H&E histological stain of tumor tissues (Mix-HD and AR101-DS2).

### DETAILED DESCRIPTION OF THE INVENTION

For the convenience of the description of the present invention, the central idea expressed in the above summary of the invention is expressed by way of specific examples. Various items in the embodiments are depicted in terms of ratios, dimensions, amounts of deformation, or displacements that are suitable for illustration, and are not drawn to the proportions of actual elements, as set forth above.

The terms “terpenoids” or “terpenes” refer to a large and diverse class of organic compounds, whose basic structure follows a general principle: 2-Methylbutane residues, less precisely but usually also referred to as isoprene units, (C₅)ₙ, build up the carbon skeleton of terpenes. About 30 000 terpenes are known at present in the literature. Depending on the number of 2-methylbutane (isoprene) subunits one differentiates between hemi-(C₅), mono-(C₁₀), sesqui-(C₁₅), di-(C₂₀), sester-(C₂₅), tri-(C₃₀), and tetraterpenes (C₄₀).

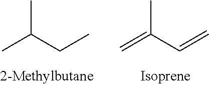

The terms “subject”, “individual”, “host”, and “patient” are used interchangeably herein to refer to a living animal, including a human and a non-human animal. The Subject may, for example, be an organism possessing immune cells capable of responding to antigenic stimulation, and stimulatory and inhibitory signal transduction through cell Surface receptor binding. The Subject may be a mammal. Such as a human or non-human mammal, for example, dogs, cats, pigs, cows, sheep, goats, horses, rats, and mice. The term ‘subject does not preclude individuals that are entirely normal with respect to a disease, or normal in all respects.

The term “treatment” refers to a therapeutic or preventative measure. The treatment may be administered to a subject having a medical disorder or who ultimately may acquire the disorder, in order to prevent, cure, delay, reduce the severity of or ameliorate one or more symptoms of a disorder or recurring disorder, or in order to prolong the Survival of a subject beyond that expected in the absence of such treatment.

The term “therapeutically effective amount” means the amount of the subject compound that may elicit a desired response, for example, a biological or medical response of a tissue, system or animal that is sought, for example, by a researcher, veterinarian, medical doctor, or other clinician.

### Cell Culture

Mouse mastocytoma cells purchased from the Bioresource Collection and Research Center (Hsinchu City, Taiwan) and were cultured with Dulbecco's modified Eagle's (DMEM) medium (Gibco, Carlsbad, California, USA) that was supplemented with 10% fetal bovine serum (Gibco, Carlsbad, California, USA), 100 U/ml penicillin, and 100 μg/ml streptomycin.

### Cell Counting Kit-8 Assay of Cell Viability

1×10⁵ cells per well of P815 cells were pretreated with terpenoids for 18 h. 5 μL Cell Counting Kit-8 reagent was added to each well, incubating for 4 h followed by optical density determination at 450 nm by a microplate reader (Tecan Sunrise). The mean optical density (OD, absorbance) from four wells of the indicated group was used to calculate the percentage of cell viability as the formula as follows: [(ODexperiment-ODblank)/(ODcontrol-ODblank)]×100%, where ODexperiment is the absorbance of cells treated with 0.1% DMSO or terpenoid-treated cells, and ODblank is the absorbance of a well with medium only. ODcontrol is the absorbance of cells treated with 0.1% DMSO only.

β-hexosaminidase Release Assay

RBL-2H3 cells were seeded into 24 well plate (0.5×10⁵ cells/0.5 mL/well), and drug treatment was added every other day for 24 hours. On the third day, 500 μL of Anti-DNP-IgE (0.1 μg/mL) was added overnight. On the fourth day, add 160 μL DNP-BSA (0.1 μg/mL in Tyrode's buffer) to each well and react at 37° C. for 1 hour. For each well in 24 wells, 50 μL of cell supernatant was mixed with 50 μL of P-NAG (triplicates), and 96 well plates were reacted at 37° C. for 1 hour. Add 100 μL of stop buffer and measure the absorbance at 405 nm, which is the β-hexosaminidase data. For 24 well plates, add 300 μL of MTT reagent (0.5 mg/mL) to each well, react at 37° C. for 2 hours, remove the supernatant, add 300 μL of DMSO to each well, and measure the absorbance at 570 nm. The data were calculated by one-way ANOVA with Dunnett's test (* p<0.5, ** p<0.01, *** p<0.001, **** p<0.0001), and plotted with Graph pad.

### Animal Experiments

All animal experiments were approved by the Laboratory Animal Management and Ethics Committee of National Chung Hsing University, Taiwan. Cultured P815 cells (1×10⁵ cells in 0.2 mL of PBS) in 200 μL of extracellular matrix gel were subcutaneously injected into the back area of female BALB/c (6-week) athymic nude mice (weight, 20+2 g). The mice were housed in a pathogen-free environment and then randomly divided into two groups for treatment (n=5) as follows: the vehicle control (10% DMSO+90% glyceryl trioctanoate) and Drug groups. The mice on both groups were daily administered via gavage feeding on the 6th day after transplantation. During the entire experimental period, the feed intake and motor activity of the mice were carefully observed, tumor sizes were measured with an electronic caliper every 3 days, and the tumor volume (mm³) was calculated according to the following formula: volume=(width²×length)/2. At the end of the study (day 20), all the mice were sacrificed by euthanization, and the tumors were quickly collected for weight measurement.

### Histopathological Analysis

Mice were sacrificed on day 30, and tumors were removed, fixed with 10% formalin, 5% formic acid, and embedded in paraffin. Briefly, tumor sections were stained with hematoxylin and eosin (H&E) and analyzed by microscopy. Tissue sections were dewaxed with Xylene, and then treated with ethanol (100%, 95%, 75%, 50%). Tissues were stained with Hematoxylin for 1 minute and Eosin for 5 minutes.

### Statistical Analysis

All data are expressed as mean ±standard deviation. GraphPad Prism 5 (GraphPad Software Inc., San Diego, CA, USA) was used to analyze statistical differences among groups by one-way ANOVA, followed by Tukey's post-test, and unpaired two-tailed t-test was performed to analyze the differences between two independent groups. A p-value <0.05 was considered as statistically significant.

### Example 1. Preparation of Antrodia Camphorata Extract

100 grams of Antrodia camphorata fruiting body is reflux with methanol for 6 hours, and the extract is collected and dried, thereby obtaining a total of 15 grams of methanol extract of Antrodia camphorata.

### Example 2. Preparation of Active Ingredients: Antcin K, Dehydrosulphurenic acid/sulphurenic acid, Versisponic acid D and Dehydroeburicoic acid

The methanol extract of Antrodia camphorata is further separated by silica column chromatography using n-hexane/ethyl acetate/methanol as eluent to provide the fractions:

| Compound | |
|---|---|
| No. | Compound name and structure |
| AR101- DS1 | 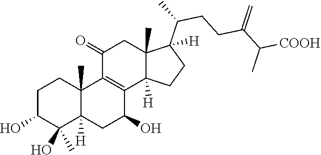 [SMILES: C=C(CC[C@@H](C)[C@H]1CC[C@H]2C3=C(C(=O)C[C@]12C)[C@@]1(C)CC[C@@H](O)[C@](C)(O)[C@@H]1C[C@@H]3O)C(C)C(=O)O] |
| | Antcin K |
| AR101- DS2 | 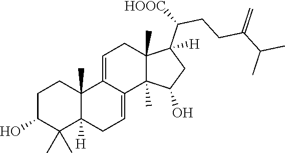 [SMILES: C=C(CC[C@@H](C(=O)O)[C@H]1C[C@H](O)[C@@]2(C)C3=CC[C@H]4C(C)(C)[C@H](O)CC[C@]4(C)C3=CC[C@]12C)C(C)C] |
| | Dehydrosulphurenic acid |
| | 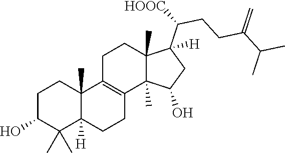 [SMILES: C=C(CC[C@@H](C(=O)O)[C@H]1C[C@H](O)[C@@]2(C)C3=C(CC[C@]12C)[C@@]1(C)CC[C@@H](O)C(C)(C)[C@@H]1CC3)C(C)C] |
| | Sulphurenic acid |
| AR101- DS3 | 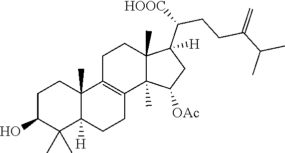 [SMILES: C=C(CC[C@@H](C(=O)O)[C@H]1C[C@H](OC(C)=O)[C@@]2(C)C3=C(CC[C@]12C)[C@@]1(C)CC[C@H](O)C(C)(C)[C@@H]1CC3)C(C)C] |
| | Versisponic acid D |
| AR101- DS4 | 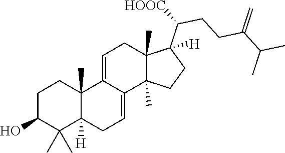 [SMILES: C=C(CC[C@@H](C(=O)O)[C@H]1CC[C@@]2(C)C3=CC[C@H]4C(C)(C)[C@@H](O)CC[C@]4(C)C3=CC[C@]12C)C(C)C] |
| | Dehydroeburicoic Acid |

### Example 3. Preparation of Active Ingredients: AR100-DS1, AR100-DS4˜ DS13

200 g of ethanol extract of anisomeles indica is taken, added into a silica-filled chromatographic column (10×15 cm), and subjected to a gradient elution with 1200 ml of each of the eluents: “n-hexane/ethyl acetate (with a ratio of 10/1, 5/1, 3/1, 1/1)”, “hexane/ethyl acetate/methanol (with a ratio of 6/4/1, 3/2/1)” and “methanol” to obtain 140 g of an initial parting liquid.

The 140 g of the initial parting liquid is separated by using silica-filled chromatographic column (10×15 cm) and subjected to a gradient elution with 1000 ml of each of the eluents: “dichloromethane”, “dichloromethane/methanol (with a ratio of 10/1, 5/1, 7/3)” and “methanol” to obtain a series of separated concentrating substance.

| Compound No. | Compound name and structure |
|---|---|
| AR100-DS1 | 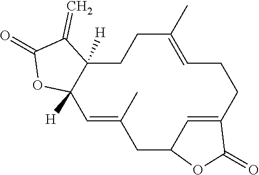 [SMILES: C=C1C(=O)O[C@H]2/C=C(\C)CC3C=C(CC/C=C(\C)CC[C@H]12)C(=O)O3] |
| | Ovatodiolide |
| AR100-DS4 | 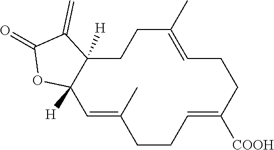 [SMILES: C=C1C(=O)O[C@H]2/C=C(\C)CC/C=C(\C(=O)O)CC/C=C(\C)CC[C@H]12] |
| | Anisomelic acid |
| AR100-DS5 | 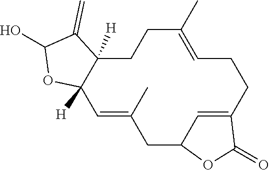 [SMILES: C=C1C(O)O[C@H]2/C=C(\C)CC3C=C(CC/C=C(\C)CC[C@H]12)C(=O)O3] |
| AR100-DS7 | 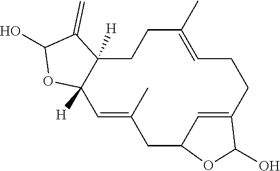 [SMILES: C=C1C(O)O[C@H]2/C=C(\C)CC3C=C(CC/C=C(\C)CC[C@H]12)C(O)O3] |
| AR100-DS8 | 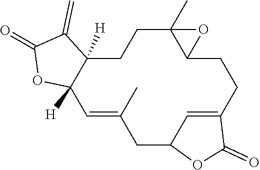 [SMILES: C=C1C(=O)O[C@H]2/C=C(\C)CC3C=C(CCC4OC4(C)CC[C@H]12)C(=O)O3] |
| AR100-DS9 | 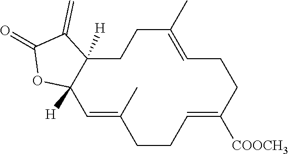 [SMILES: C=C1C(=O)O[C@H]2/C=C(\C)CC/C=C(\C(=O)OC)CC/C=C(\C)CC[C@H]12] |
| AR100-DS10 | 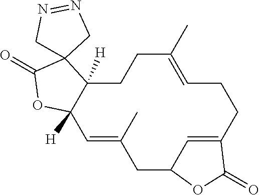 [SMILES: C/C1=C\CCC2=CC(C/C(C)=C/[C@@H]3OC(=O)C4(CN=NC4)[C@H]3CC1)OC2=O] |
| AR100-DS11 | 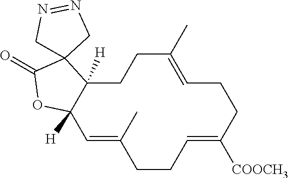 [SMILES: COC(=O)/C1=C\CC/C(C)=C/[C@@H]2OC(=O)C3(CN=NC3)[C@H]2CC/C(C)=C/CC1] |
| AR100-DS12 | 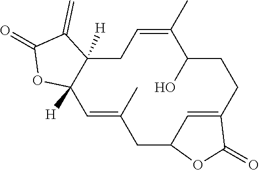 [SMILES: C=C1C(=O)O[C@H]2/C=C(\C)CC3C=C(CCC(O)/C(C)=C\C[C@H]12)C(=O)O3] |

### Example 4. Effects of Terpenoids on the Cell Viability on RBL-2H3 Cells and the Inhibition Effect on β-Hexosaminidase Release

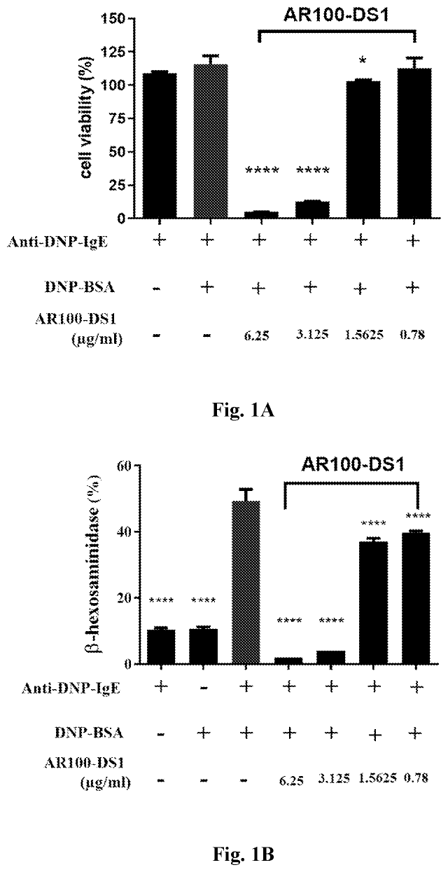

As shown in FIGS. 1, under the safety dose, a significantly lower of β-hexosaminidase release was observed in AR100-DS1-treated groups. The results from triplicate determinations are presented as the percentage of viable cells. Statistical analysis was performed using one-way ANOVA (Dunnett's multiple comparisons test). Results are presented as the mean ±SD from at least three independent experiments. Statistical significance is indicated (ns: no significance, ** p<0.01, *** p<0.001, **** p<0.0001, compared with DMSO).

### Example 5. Effects of Terpenoids on the Cell Viability in RBL-2H3 Cells

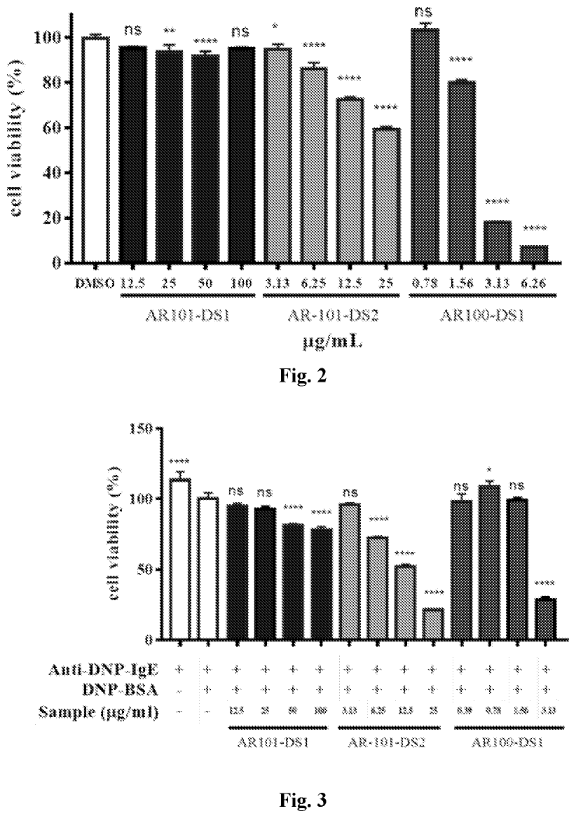

As shown in FIG. 2, the cell viability was determined using MTT assay. The results from triplicate determinations are presented as the percentage of viable cells. Statistical analysis was performed using one-way ANOVA (Dunnett's multiple comparisons test). Results are presented as the mean ±SD from at least three independent experiments. Statistical significance is indicated (ns: no significance, ** p<0.01, *** p<0.001, **** p<0.0001, compared with DMSO).

### Example 6. Effects of Terpenoids on the Cell Viability and the β-Hexosaminidase Release in RBL-2H3 Cells

As shown in FIG. 3, the cell viability was determined using MTT assay. The results from triplicate determinations are presented as the percentage of viable cells. Statistical analysis was performed using one-way ANOVA (Dunnett's multiple comparisons test). Results are presented as the mean ±SD from at least three independent experiments. Statistical significance is indicated (ns: no significance, ** p<0.01, *** p<0.001, **** p<0.0001, compared with DMSO).

### Example 7. Effects of Terpenoids on the B-Hexosaminidase Release in RBL-2H3 Cells

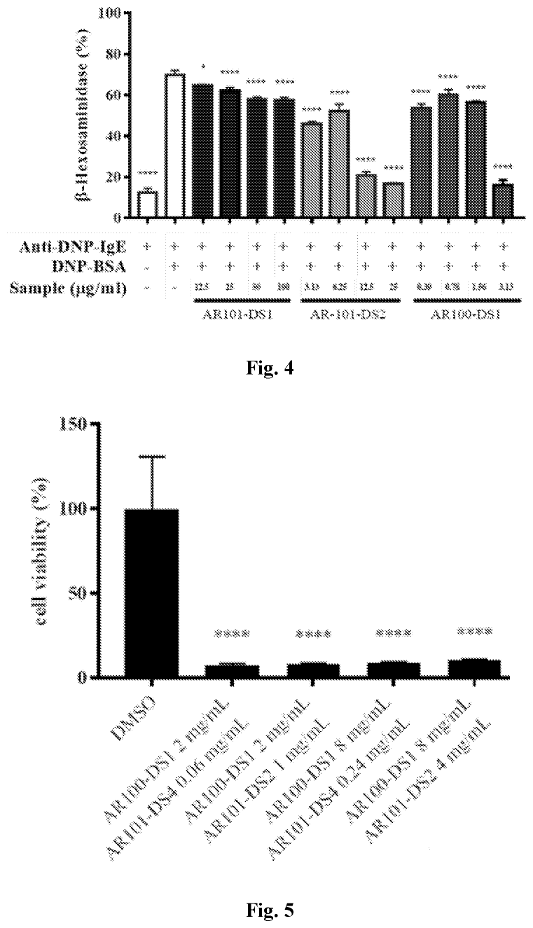

As shown in FIG. 4, the results from triplicate determinations are presented as the percentage of maximal antigen-induced degranulation. Statistical analysis was performed using one-way ANOVA (Dunnett's multiple comparisons test). Results are presented as the mean ±SD from at least three independent experiments. Statistical significance is indicated (ns: no significance, ** p<0.01, p<0.001, **** p<0.0001, compared with DMSO).

### Example 8. Effects of Terpenoids on the Cell Viability on P815 Cells

Mouse mastocytoma cells purchased from the Bioresource Collection and Research Center (Hsinchu City, Taiwan) and were cultured with Dulbecco's modified Eagle's (DMEM) medium (Gibco, Carlsbad, California, USA) that was supplemented with 10% fetal bovine serum (Gibco, Carlsbad, California, USA), 100 U/ml penicillin, and 100 μg/ml streptomycin. 1×10⁵ cells per well of P815 cells were pretreated with terpenoids for 18 h. 5 μL Cell Counting Kit-8 reagent was added to each well, incubating for 4 h followed by optical density determination at 450 nm by a microplate reader (Tecan Sunrise). The mean optical density (OD, absorbance) from four wells of the indicated group was used to calculate the percentage of cell viability as the formula as follows: [(ODexperiment-ODblank)/(ODcontrol-ODblank)]×100%, where ODexperiment is the absorbance of cells treated with 0.1% DMSO or terpenoids-treated cells, and ODblank is the absorbance of a well with medium only. ODcontrol is the absorbance of cells treated with 0.1% DMSO only.

As shown in FIG. 5, according to the study result, effects of these terpenoids or combination groups composed by terpenoids show significant inhibition ability of mast cancer cell P815, combination groups especially.

| | Cell viability (%) | p- |
|---|---|---|
| Groups | Mean ± SD | value |
| DMSO | 100.00 ± 9.38 | |
| AR100-DS1 1 mg/mL | 42.44 ± 1.84 | **** |
| AR100-DS1 2 mg/mL | 39.52 ± 3.09 | **** |
| AR100-DS1 3 mg/mL | 40.81 ± 1.25 | **** |
| AR100-DS1 4 mg/mL | 43.33 ± 0.65 | **** |
| AR100-DS1 5 mg/mL | 41.39 ± 1.81 | **** |
| AR101-DS2 0.25 mg/mL | 265.98 ± 28.34 | **** |
| AR101-DS2 0.5 mg/mL | 130.20 ± 12.98 | ns |
| AR101-DS2 0.75 mg/mL | 80.75 ± 5.85 | ns |
| AR101-DS4 0.02 mg/mL | 181.35 ± 30.40 | **** |
| AR101-DS4 0.04 mg/mL | 69.27 ± 4.58 | ns |
| AR101-DS4 0.06 mg/mL | 49.70 ± 2.62 | ** |
| AR101-DS4 0.08 mg/mL | 54.29 ± 1.85 | ** |
| AR101-DS4 0.1 mg/mL | 49.15 ± 2.52 | ** |

| | | | Cell viability (%) | |
|---|---|---|---|---|
| | Seeding 1 × 10⁵ P815 | | Mean ± SD | p-value |
| DMSO | | | 100.00 ± 31.11 | |
| | AR100-DS1 | AR101-DS4 | 7.65 ± 1.01 | **** |
| | 2 mg/mL | 0.06 mg/mL | | |
| | AR100-DS1 | AR101-DS2 | 8.13 ± 0.85 | **** |
| | 2 mg/mL | 1 mg/mL | | |
| | AR100-DS1 | AR101-DS4 | 9.07 ± 0.68 | **** |
| | 8 mg/mL | 0.24 mg/mL | | |
| | AR100-DS1 | AR101-DS2 | 10.59 ± 0.57 | **** |
| | 8 mg/mL | 4 mg/mL | | |

### Example 10. The Effect of Terpenoids Administration on P815 Cell Development in Nude Mice

Male BALB/c mice are divided into the following groups:
1. Vehicle (10% DMSO+90% Olive oil)
2. AR100-DS1 (2 mg/kg)
3. AR100-DS1 (7.5 mg/kg)
4. AR100-DS1 (2 mg/kg)+AR101-DS4 (0.1 mg/kg)
5. AR100-DS1 (7.5 mg/kg)+AR101-DS4 (1 mg/kg)
6. AR101-DS4 (0.06 mg/kg)
7. AR101-DS4 (0.24 mg/kg)
8. AR101-DS2 (1 mg/kg)
9. AR101-DS4 (4 mg/kg)

The mice were administrated 100 μL samples intravenously every 2 days and sacrificed on the 30th day for histopathological examinations.

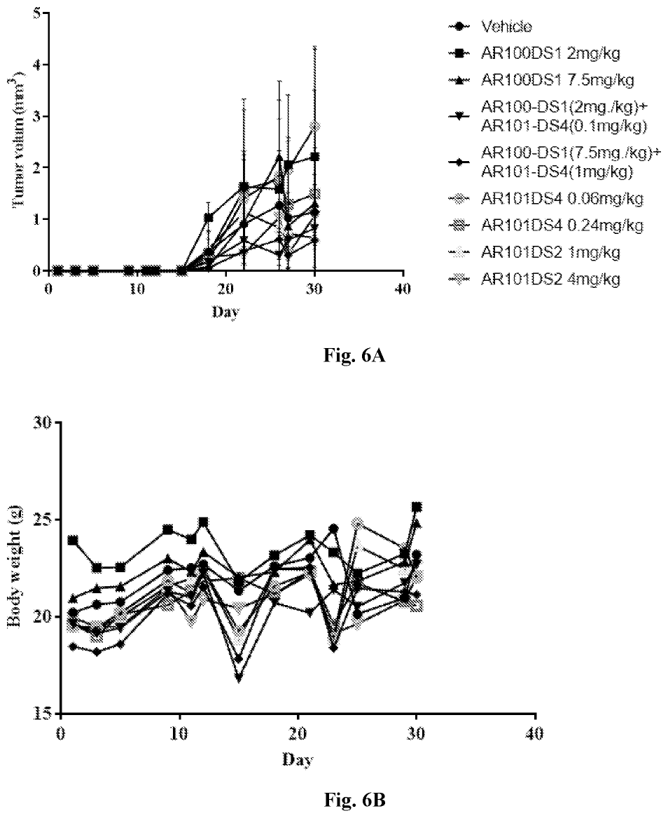

As shown in FIG. 6A, comparing to the volume change of P815 mast cell tumor of the Vehicle group, these following groups: AR100-DS1+AR101-DS4 low dose, AR100-DS1+AR101-DS4 high dose, AR101-DS2 1 mg/kg and AR101-DS2 4 mg/kg, significantly reduced tumor growth was observed.

As shown in FIG. 6B, the mean body weight over time curve. All groups of this study show no significant change of animals' body weight.

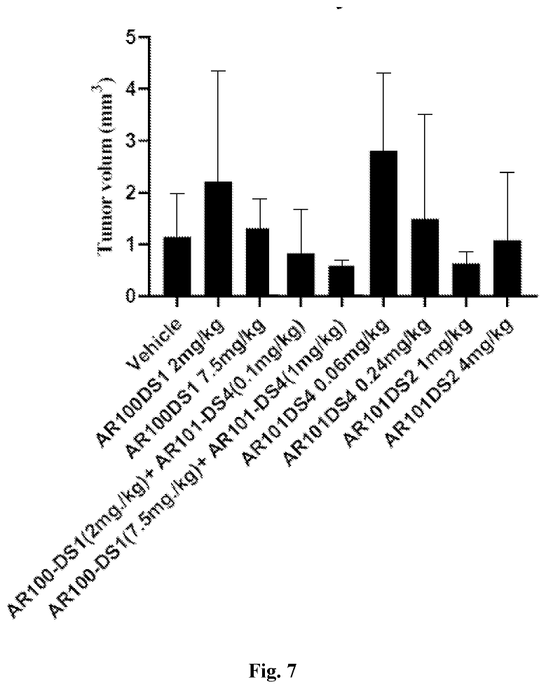

As shown in FIG. 7, Comparing to the volume change of P815 mast cell tumor of the Vehicle group, these following groups: Mix low dose, Mix high dose, AR101-DS2 1 mg/kg and AR101-DS2 4 mg/kg, significantly reduced tumor growth was observed.

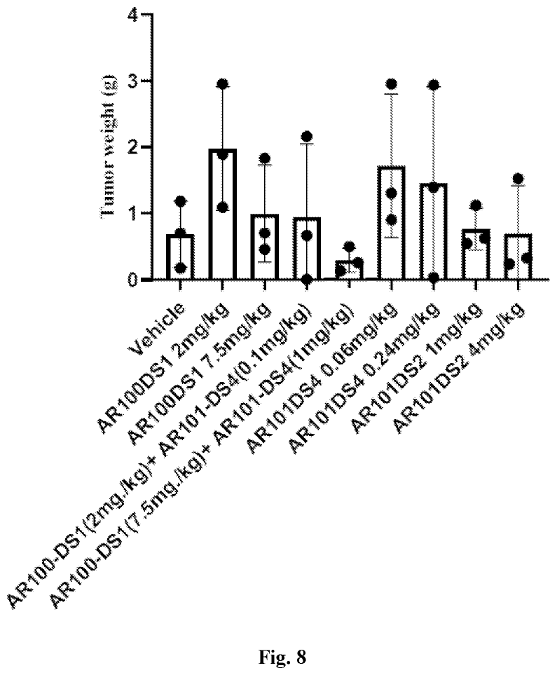

As shown in FIG. 8, Per our study result, Mix HD inhibit the mast cell tumor growth significantly. According to the statistical analysis result standard deviation of Mix HD is quiet low, that means terpenoids show strong potential for treating the mast cell cancer.

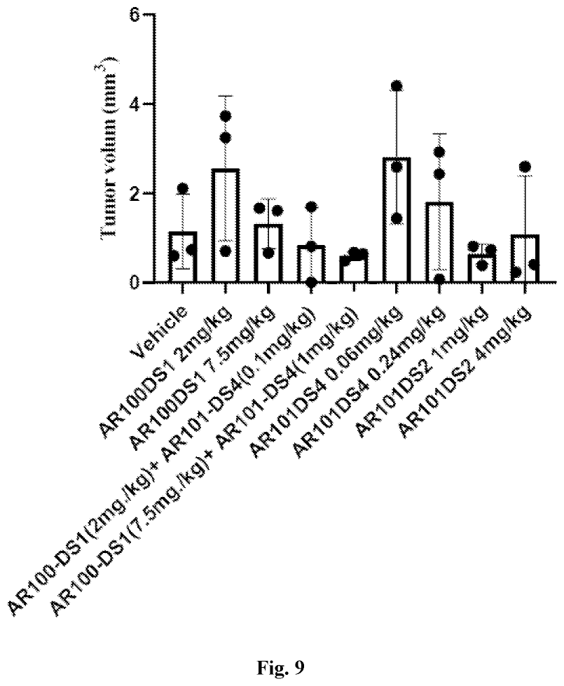

As shown in FIG. 9, per our study result, Mix LD, Mix HD and AR101-DS2 1 mg/kg inhibit the mast cell tumor growth significantly. According to the statistical analysis result standard deviation of Mix HD and AR101-DS2 are quiet low, that means terpenoids show strong potential for treating the mast cell cancer.

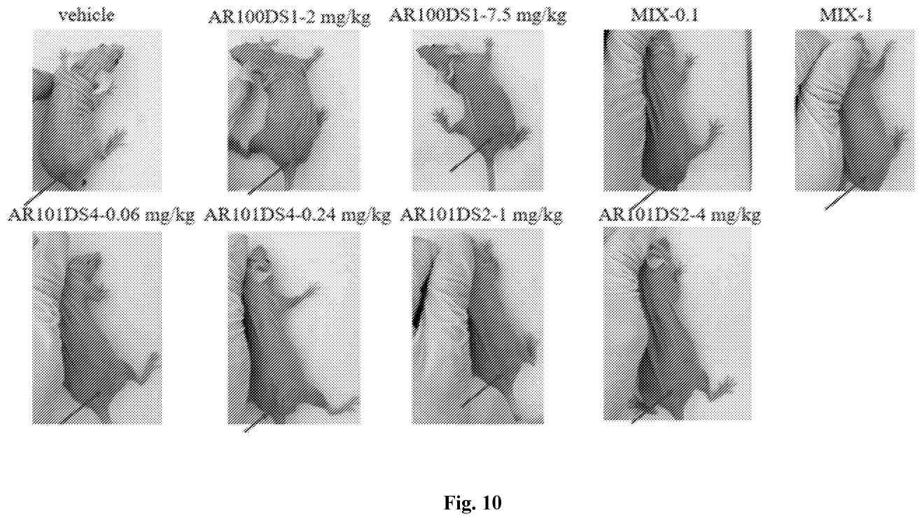

As shown in FIG. 10, the P815 mast cell tumor was located in right rear side near the tail. Comparing to the tumor's volume change of Vehicle group, these following groups: Mix LD, Mix HD, AR101-DS2 1 mg/kg and AR101-DS2 4 mg/kg, significantly reduced tumor growth was observed.

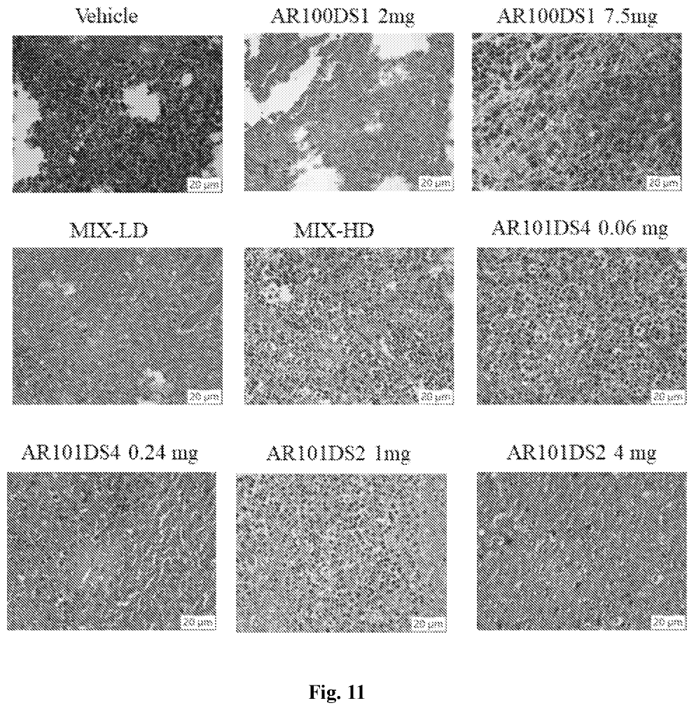

As shown in FIG. 11, MIX-HD, AR101-DS4 0.06 mg and AR101-DS2 1 mg groups show their ability to arrest the tumor cell proliferation and cause the tumor cell nucleus aggregation. In this circumstance these processes maybe induced by cell apoptosis.

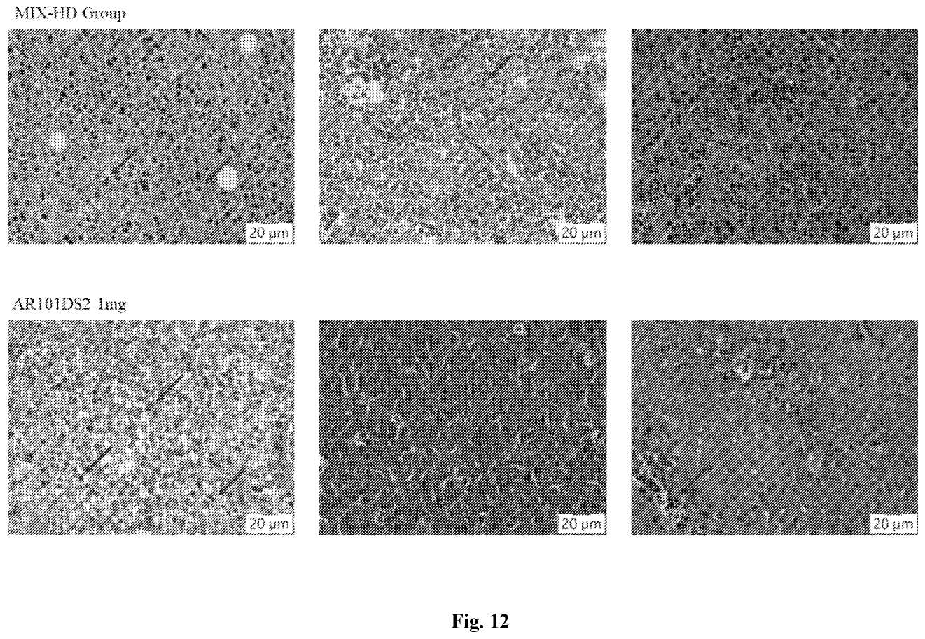

As shown in FIG. 12, the Mix-HD and AR101DS2 1 mg/kg groups arrest the tumor cell proliferation, cause the tumor cell nucleus aggregation, and cell to cell connection lost their tightness.

What is claimed is:

1. A method of treating mastocytoma, comprising administering an effective amount of composition to a subject in need thereof; wherein the subject is a non-human animal; wherein the composition comprises at least one compound selected from the group consisting following formulae and combinations thereof:

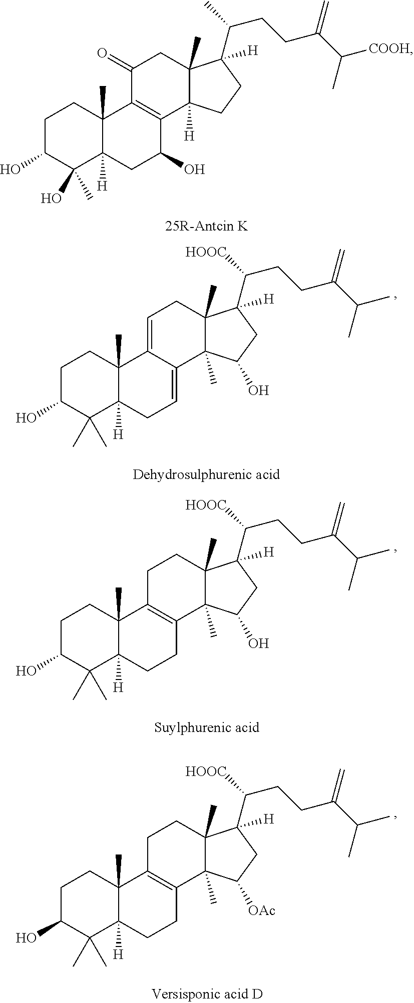 [SMILES: C=C(CC[C@@H](C)[C@H]1CC[C@H]2C3=C(C(=O)C[C@]12C)[C@@]1(C)CC[C@@H](O)[C@](C)(O)[C@@H]1C[C@@H]3O)C(C)C(=O)O ; C=C(CC[C@@H](C(=O)O)[C@H]1C[C@H](O)[C@@]2(C)C3=CC[C@H]4C(C)(C)[C@H](O)CC[C@]4(C)C3=CC[C@]12C)C(C)C ; C=C(CC[C@@H](C(=O)O)[C@H]1C[C@H](O)[C@@]2(C)C3=C(CC[C@]12C)[C@@]1(C)CC[C@@H](O)C(C)(C)[C@@H]1CC3)C(C)C ; C=C(CC[C@@H](C(=O)O)[C@H]1C[C@H](OC(C)=O)[C@@]2(C)C3=C(CC[C@]12C)[C@@]1(C)CC[C@H](O)C(C)(C)[C@@H]1CC3)C(C)C]

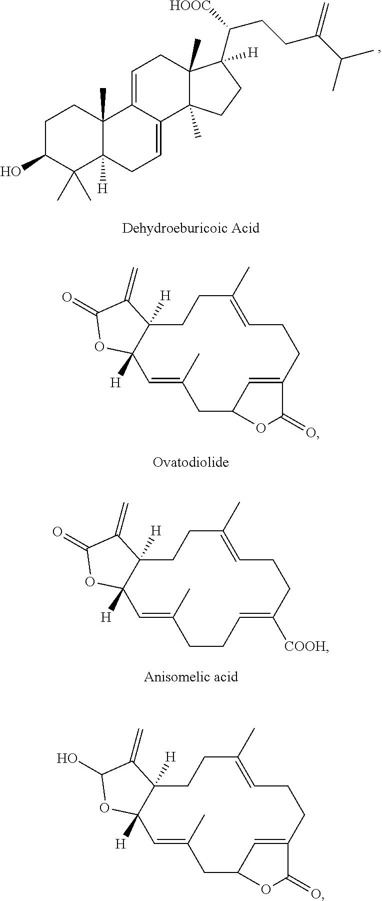 [SMILES: C=C(CC[C@@H](C(=O)O)[C@H]1CC[C@@]2(C)C3=CC[C@H]4C(C)(C)[C@@H](O)CC[C@]4(C)C3=CC[C@]12C)C(C)C ; C=C1C(=O)O[C@H]2/C=C(\C)CC3C=C(CC/C=C(\C)CC[C@H]12)C(=O)O3 ; C=C1C(=O)O[C@H]2/C=C(\C)CC/C=C(\C(=O)O)CC/C=C(\C)CC[C@H]12 ; C=C1C(O)O[C@H]2/C=C(\C)CC3C=C(CC/C=C(\C)CC[C@H]12)C(=O)O3]

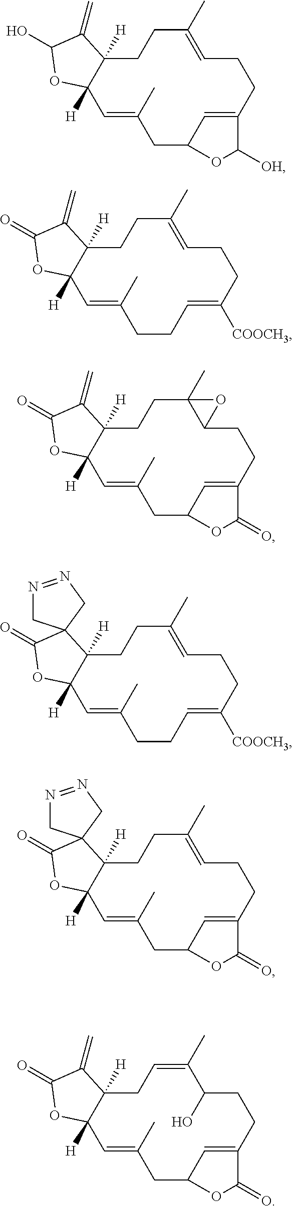

2. The method of claim 1, wherein the composition comprises:
(a) 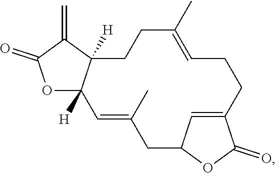 [SMILES: C=C1C(=O)O[C@H]2/C=C(\C)CC3C=C(CC/C=C(\C)CC[C@H]12)C(=O)O3]
and
(b) 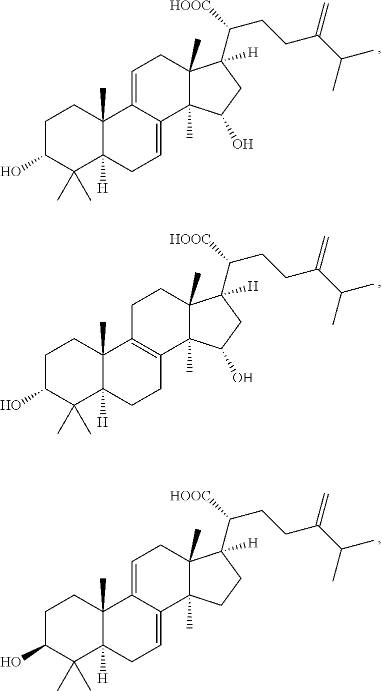 [SMILES: C=C(CC[C@@H](C(=O)O)[C@H]1C[C@H](O)[C@@]2(C)C3=CC[C@H]4C(C)(C)[C@H](O)CC[C@]4(C)C3=CC[C@]12C)C(C)C ; C=C(CC[C@@H](C(=O)O)[C@H]1C[C@H](O)[C@@]2(C)C3=C(CC[C@]12C)[C@@]1(C)CC[C@@H](O)C(C)(C)[C@@H]1CC3)C(C)C ; C=C(CC[C@@H](C(=O)O)[C@H]1CC[C@@]2(C)C3=CC[C@H]4C(C)(C)[C@@H](O)CC[C@]4(C)C3=CC[C@]12C)C(C)C]
or combinations thereof.

3. The method of claim 2, wherein the composition comprises:

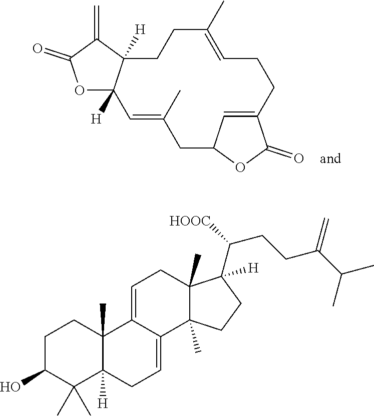 [SMILES: C=C1C(=O)O[C@H]2/C=C(\C)CC3C=C(CC/C=C(\C)CC[C@H]12)C(=O)O3 ; C=C(CC[C@@H](C(=O)O)[C@H]1CC[C@@]2(C)C3=CC[C@H]4C(C)(C)[C@@H](O)CC[C@]4(C)C3=CC[C@]12C)C(C)C]

4. A composition, comprises:
(a) 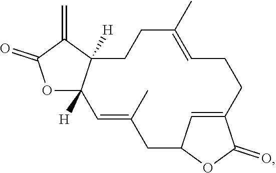 [SMILES: C=C1C(=O)O[C@H]2/C=C(\C)CC3C=C(CC/C=C(\C)CC[C@H]12)C(=O)O3]
and
(b) 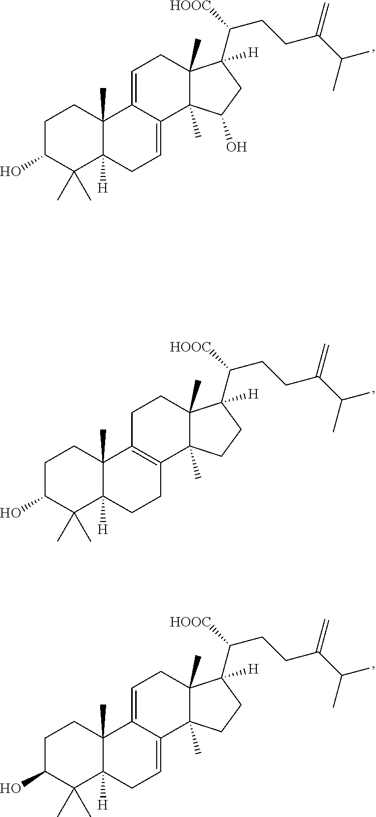
or combinations thereof.

5. The composition of claim 4, which comprises: [chemical structure]
